(19) United States
(12) Patent Application Publication
(10) Pub. No.: US 2022/0304555 A1
(43) Pub. Date: Sep. 29, 2022

(54) SYSTEMS AND METHODS FOR USE OF STEREOSCOPY AND COLOR CHANGE MAGNIFICATION TO ENABLE MACHINE LEARNING FOR MINIMALLY INVASIVE ROBOTIC SURGERY

(71) Applicant: Covidien LP, Mansfield, MA (US)

(72) Inventors: Dwight Meglan, Westwood, MA (US); Meir Rosenberg, Newton, MA (US); Joshua Reed, Saint Paul, MN (US)

(21) Appl. No.: 17/638,903

(22) PCT Filed: Oct. 1, 2020
(86) PCT No.: PCT/US2020/053790

§ 371 Date: Feb. 28, 2022

Related U.S. Application Data
(60) Provisional application No. 62/910,514, filed on Oct. 4, 2019.

(51) Int. Cl. A61B 1/00; A61B 34/20; H04N 13/128; G06T 7/593; G16H 30/40

(52) U.S. Cl. CPC A61B 1/000095; A61B 1/000096; A61B 1/000094; A61B 34/20; H04N 13/128; G06T 7/593; G16H 30/40; A61B 1/00045; A61B 2034/2065

(57) ABSTRACT

A computer-implemented method of object enhancement in endoscopy images is presented. The computer-implemented method includes capturing an image of an object within a surgical operative site, by an imaging device. The image includes a plurality of pixels. Each of the plurality of pixels includes color information. The computer-implemented method further includes accessing the image, accessing data relating to depth information about each of the pixels in the image, inputting the depth information to a machine learning algorithm, emphasizing a feature of the image based on an output of the neural network, generating an augmented image based on the emphasized feature, and displaying the augmented image on a display.

### FIELD

The present disclosure relates to devices, systems, and methods for surgical tool identification in images, and more particularly, to enhancing aspects of discernable features of objects during surgical procedures.

### BACKGROUND

Endoscopes are introduced through an incision or a natural body orifice to observe internal features of a body. Conventional endoscopes are used for visualization during endoscopic or laparoscopic surgical procedures. During such surgical procedures, it is possible for the view of the instrument to be obstructed by tissue or other instruments.

During minimally invasive surgery, and especially in robotic surgery, knowledge of the exact surgical tools appearing in the endoscopic video feed can be useful for facilitating features that enhance the surgical experience. While electrical or wireless communication between something attached/embedded in the tool is a possible means to do this, when this infrastructure is either not available or not possible, another identification means is needed. Accordingly, there is interest in improving imaging technology.

### SUMMARY

The disclosure relates to devices, systems, and methods for surgical tool identification in images. In accordance with aspects of the disclosure, a system for object enhancement in endoscopy images is presented. The system includes a light source, an imaging device, and an imaging device control unit. The light source is configured to provide light within a surgical operative site. The imaging device control unit includes a processor and a memory storing instructions. The instructions, when executed by the processor, cause the system to capture an image of an object within the surgical operative site, by the imaging device. The image includes a plurality of pixels. Each of the plurality of pixels includes color information. The instructions, when executed by the processor, further cause the system to access the image, access data relating to depth information about each of the pixels in the image, input the depth information to a neural network, emphasize a feature of the image based on an output of the machine learning algorithm, generate an augmented image based on the emphasized feature, and display the augmented image on a display.

In an aspect of the present disclosure, emphasizing the feature may include augmenting a 3D aspect of the image, emphasizing a boundary of the object, changing the color information of the plurality of pixels of the object, and/or extracting 3D features of the object.

In another aspect of the present disclosure, the instructions, when executed, may further cause the system to perform real-time image recognition on the augmented image to detect an object and classify the object.

In an aspect of the present disclosure, the image may include a stereographic image. The stereographic image may include a left image and a right image. The instructions, when executed, may further cause the system to calculate depth information based on determining a horizontal disparity mismatch between the left image and the right image. The depth information may include pixel depth.

In yet another aspect of the present disclosure, the instructions, when executed, may further cause the system to calculate depth information based on structured light projection. The depth information may include pixel depth.

In a further aspect of the present disclosure, the machine learning algorithm may include a convolutional neural network, a feed forward neural network, a radial bias neural network, a multilayer perceptron, a recurrent neural network, and/or a modular neural network.

In an aspect of the present disclosure, the machine learning algorithm may be trained based on tagging objects in training images. The training may further include augmenting the training images to include adding noise, changing colors, hiding portions of the training images, scaling of the training images, rotating the training images, and/or stretching the training images.

In a further aspect of the present disclosure, the training may include supervised, unsupervised, and/or reinforcement learning.

In yet another aspect of the present disclosure, the instructions, when executed, may further cause the system to: process a time series of the augmented image based on a learned video magnification, phase-based video magnification, and/or Eulerian video magnification.

In a further aspect of the present disclosure, the instructions, when executed, may further cause the system to perform tracking of the object based on an output of the machine learning algorithm.

In accordance with aspects of the disclosure, a computer-implemented method of object enhancement in endoscopy images is presented. The method includes capturing an image of an object within a surgical operative site, by an imaging device. The image includes a plurality of pixels. Each of the plurality of pixels includes color information. The method further includes accessing the image, accessing data relating to depth information about each of the pixels in the image, inputting the depth information to a machine learning algorithm, emphasizing a feature of the image based on an output of the machine learning algorithm, generating an augmented image based on the emphasized feature, and displaying the augmented image on a display.

In an aspect of the present disclosure, emphasizing the feature may include augmenting a 3D aspect of the image, emphasizing a boundary of the object, changing the color information of the plurality of pixels of the object, and/or extracting 3D features of the object.

In yet a further aspect of the present disclosure, the computer-implemented method may further include performing real-time image recognition on the augmented image to detect an object and classify the object.

In yet another aspect of the present disclosure, the image may include a stereographic image. The stereographic image may include a left image and a right image. The computer-implemented method may further include calculating depth information based on determining a horizontal disparity mismatch between the left image and the right image. The depth information may include pixel depth.

In a further aspect of the present disclosure, the computer-implemented method may further include calculating depth information based on structured light projection. The depth information may include pixel depth.

In yet a further aspect of the present disclosure, the machine learning algorithm may include a convolutional neural network, a feed forward neural network, a radial bias neural network, a multilayer perceptron, a recurrent neural network, and/or a modular neural network.

In yet another aspect of the present disclosure, the machine learning algorithm may be trained based on tagging objects in training images. The training may further include augmenting the training images to include adding noise, changing colors, hiding portions of the training images, scaling of the training images, rotating the training image, and/or stretching the training images.

In a further aspect of the present disclosure, the computer-implemented method may further include processing a time series of the augmented image based on a learned video magnification, phase-based video magnification, and/or Eulerian video magnification.

In an aspect of the present disclosure, the computer-implemented method may further include performing tracking of the object based on an output of the machine learning algorithm.

In accordance with aspects of the present disclosure, a non-transitory storage medium that stores a program causing a computer to execute a computer-implemented method of object enhancement in endoscopy images is presented. The computer-implemented method includes capturing an image of an object within a surgical operative site, by an imaging device. The image includes a plurality of pixels, each of the plurality of pixels includes color information. The method further includes accessing the image, accessing data relating to depth information about each of the pixels in the image, inputting the depth information to a machine learning algorithm, emphasizing a feature of the image based on an output of the machine learning algorithm, generating an augmented image based on the emphasized feature, and displaying the augmented image on a display.

In accordance with aspects of the present disclosure, a system for object detection in endoscopy images is presented. The system includes a light source configured to provide light within a surgical operative site, an imaging device configured to acquire stereographic images, and an imaging device control unit configured to control the imaging device. The control unit includes a processor and a memory storing instructions. The instructions, when executed by the processor, cause the system to: capture a stereographic image of an object within a surgical operative site, by the imaging device. The stereographic image includes a first image and a second image. The instructions, when executed by the processor, further cause the system to: access the stereographic image, perform real time image recognition on the first image to detect the object, classify the object, and produce a first image classification probability value, perform real time image recognition on the second image to detect the object, classify the object, and produce a first image classification probability value, and compare the first image classification probability value and the second image classification probability value to produce a classification accuracy value. In a case where the classification probability value is above a predetermined threshold, the instructions, when executed by the processor, further cause the system to: generate a first bounding box around the detected object, generate a first augmented view of the first image based on the classification, generate a second augmented view of the second image based on the classification, and display the first and second augmented images on a display. The first augmented view includes the bounding box and a tag indicating the classification. The second augmented view includes the bounding box and a tag indicating the classification.

In an aspect of the present disclosure, in a case where the classification accuracy value is below the predetermined threshold, the instructions, when executed, may further cause the system to display on the display an indication that the classification accuracy value is not within an expected range.

In another aspect of the present disclosure, the real-time image recognition may include: detecting the object in the first image, detecting the object in the second image, generating a first silhouette of the object in the first image, generating a second silhouette of the object in the second image, comparing the first silhouette to the second silhouette, and detecting inconsistencies between the first silhouette and the second silhouette based on the comparing.

In an aspect of the present disclosure, the real-time image recognition may include: detecting the object based on a convolutional neural network. In various The detecting may include generating a segmentation mask for the object, detecting the object, and classifying the object based on the detecting.

In yet another aspect of the present disclosure, the convolutional neural network may be trained based on tagging objects in training images, and wherein the training further includes augmenting the training images to include adding noise, changing colors, hiding portions of the training images, scaling of the training images, rotating the training image, and/or stretching the training images.

In a further aspect of the present disclosure, the real-time image recognition may include detecting the object based on a region based neural network. The detecting may include dividing the first image and second image into regions, predicting bounding boxes for each region based on a feature of the object, predicting an object detection probability for each region, weighting the bounding boxes based on the predicted object detection probability, detecting the object, and classifying the object based on the detecting.

In an aspect of the present disclosure, the region based neural network may be trained based on tagging objects in training images, and wherein the training further includes augmenting the training images to include adding noise, changing colors, hiding portions of the training images, scaling of the training images, rotating the training images, changing a background, and/or stretching the training images.

In a further aspect of the present disclosure, the instructions, when executed, may further cause the system to: perform tracking of the object based on an output of the region based neural network.

In yet another aspect of the present disclosure, the first and second augmented views each may further include an indication of the classification accuracy value.

In accordance with aspects of the present disclosure, a computer-implemented method of object detection in endoscopy images is presented. The computer-implemented method includes accessing a stereographic image of an object within a surgical operative site, by an imaging device. The stereographic image includes a first image and a second image. The method further includes performing real-time image recognition on the first image to detect the object and classify the object performing real-time image recognition on the second image to detect the object, classify the object, and produce a classification probability value, and comparing the classification probability value of the first image and the classification probability value of the second image based on the real-time image recognition to produce a classification accuracy value. In a case where the classification accuracy value is above a predetermined threshold, the method further includes generating a first bounding box around the detected object, generating a first augmented view of the first image based on the classification generating a second augmented view of the second image based on the classification the bounding box, and displaying the first and second augmented images on a display. The first augmented view includes the bounding box and a tag indicating the classification. The second augmented view includes the bounding box and a tag indicating the classification.

In a further aspect of the present disclosure, in a case where the classification accuracy value is below the predetermined threshold, the method may further include displaying on the display an indication that the classification accuracy value is not within an expected range.

In yet a further aspect of the present disclosure, the real-time image recognition may include detecting the object in the first image, detecting the object in the second image, generating a first silhouette of the object in the first image, generating a second silhouette of the object in the second image, comparing the first silhouette to the second silhouette, and detecting inconsistencies between the first silhouette and the second silhouette based on the comparing.

In yet another aspect of the present disclosure, the real-time image recognition may include detecting the object based on a convolutional neural network. The detecting may include generating a segmentation mask for the object, detecting the object, and classifying the object based on the detecting.

In a further aspect of the present disclosure, the convolutional neural network may be trained based on tagging objects in training images. The training may further include augmenting the training images to include adding noise, changing colors, hiding portions of the training images, scaling of the training images, rotating the training images, and/or stretching the training images.

In yet a further aspect of the present disclosure, the real-time image recognition may include detecting the object based on a region based neural network. The detecting may include diving the image into regions, predicting bounding boxes for each region based on a feature of the object, predicting an object detection probability for each region, weighting the bounding boxes based on the predicted object detection probability, detecting the object, and classifying the object based on the detecting.

In yet another aspect of the present disclosure, the region based neural network may be trained based on tagging objects in training images. The training may further include augmenting the training images to include adding noise, changing colors, hiding portions of the training images, scaling of the training images, rotating the training images, changing background, and/or stretching the training images.

In a further aspect of the present disclosure, the method may further include performing tracking of the object based on an output of the region based neural network.

In an aspect of the present disclosure, the first and second augmented views each may further include an indication of the classification probability value.

In accordance with aspects of the present disclosure, a non-transitory storage medium that stores a program causing a computer to execute a computer-implemented method of object enhancement in endoscopy images is presented. The computer-implemented method includes accessing a stereographic image of an object within a surgical operative site, by an imaging device.

The stereographic image includes a first image and a second image. The computer-implemented method further includes performing real-time image recognition on the first image to detect the object and classify the object performing real-time image recognition on the second image to detect the object, classify the object, and produce a classification probability value, and comparing the classification probability value of the first image and the classification probability value of the second image based on the real-time image recognition to produce a classification accuracy value. In a case where the classification accuracy value is above a predetermined threshold, the method further includes generating a first bounding box around the detected object, generating a first augmented view of the first image based on the classification, generating a second augmented view of the second image based on the classification the bounding box, and displaying the first and second augmented images on a display. The first augmented view includes the bounding box and a tag indicating the classification. The second augmented view includes the bounding box and a tag indicating the classification.

Further details and aspects of various embodiments of the disclosure are described in more detail below with reference to the appended figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the disclosure are described herein with reference to the accompanying drawings, wherein:

FIG. 1 is a diagram of an exemplary visualization or endoscope system in accordance with the disclosure;

FIG. 2 is a schematic configuration of the visualization or endoscope system of FIG. 1;

FIG. 3 is a diagram illustrating another schematic configuration of an optical system of the system of FIG. 1;

FIG. 4 is a schematic configuration of the visualization or endoscope system in accordance with an embodiment of the disclosure;

FIG. 5 is a flowchart of a method for object enhancement in endoscopy images in accordance with an exemplary embodiment of the disclosure;

FIG. 6A is an exemplary input image in accordance with the disclosure;

FIG. 6B is an exemplary output image with the subject's pulse signal amplified in accordance with the disclosure;

FIG. 6C is an exemplary vertical scan line from the output image of FIG. 6B;

FIG. 6D is an exemplary vertical scan line from the input image of FIG. 6A;

FIG. 7 is a flowchart of a method for object detection in endoscopy images in accordance with an exemplary embodiment of the disclosure;

FIG. 8 is an exemplary input image in accordance with the disclosure;

FIG. 9 is an exemplary output image in accordance with the disclosure;

FIG. 10 is first and second augmented images in accordance with the disclosure;

FIG. 11 is a diagram of an exemplary process for real-time image detection in accordance with the disclosure; and

FIG. 12 is a diagram of a region proposal network for real-time image detection in accordance with the disclosure.

Further details and aspects of exemplary embodiments of the disclosure are described in more detail below with reference to the appended figures. Any of the above aspects and embodiments of the disclosure may be combined without departing from the scope of the disclosure.

### DETAILED DESCRIPTION OF EMBODIMENTS

Embodiments of the presently disclosed devices, systems, and methods of treatment are described in detail with reference to the drawings, in which like reference numerals designate identical or corresponding elements in each of the several views. As used herein, the term “distal” refers to that portion of a structure that is farther from a user, while the term “proximal” refers to that portion of a structure that is closer to the user. The term “clinician” refers to a doctor, nurse, or other care provider and may include support personnel.

The disclosure is applicable where images of a surgical site are captured. Endoscope systems are provided as an example, but it will be understood that such description is exemplary and does not limit the scope and applicability of the disclosure to other systems and procedures.

Convolutional neural network-based machine learning may be used in conjunction with minimally invasive endoscopic surgical video for surgically useful purposes, such as discerning potentially challenging situations, which requires that the networks be trained on clinical video. The anatomy seen in these videos can be complex as well as subtle and the surgical tool interaction with the anatomy equally challenging to yield the details of the interaction. Means by which the actions observed are enhanced/emphasized would be desirable to assist the machine learning to yield better insights with less training.

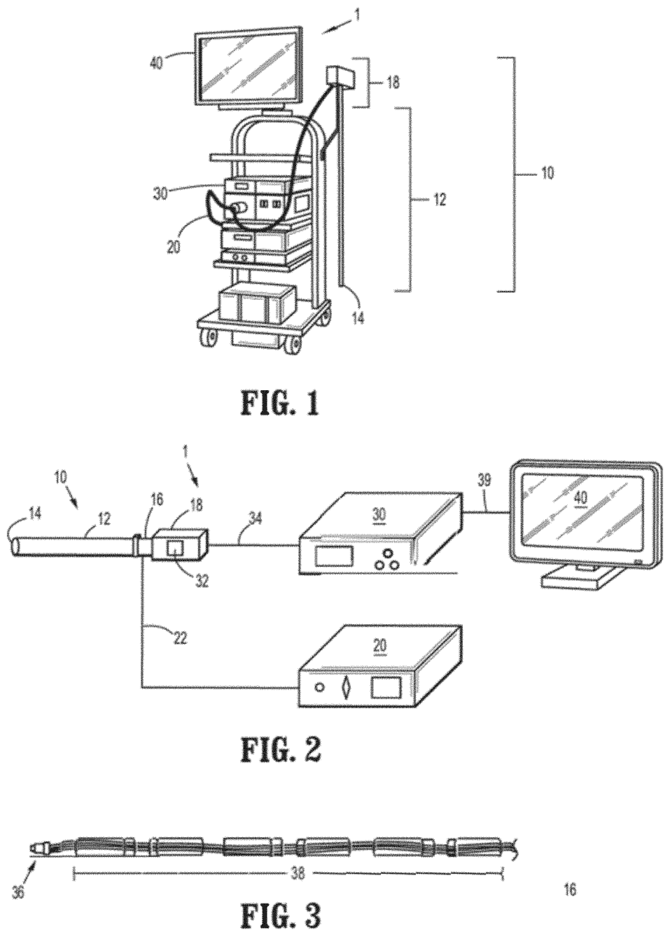

Referring initially to FIGS. 1-3, an endoscope system 1, in accordance with the disclosure, includes an endoscope 10, a light source 20, a video system 30, and a display device 40. With continued reference to FIG. 1, the light source 20, such as an LED/Xenon light source, is connected to the endoscope 10 via a fiber guide 22 that is operatively coupled to the light source 20 and to an endocoupler 16 disposed on, or adjacent to, a handle 18 of the endoscope 10. The fiber guide 22 includes, for example, fiber optic cable which extends through the elongated body 12 of the endoscope 10 and terminates at a distal end 14 of the endoscope 10. Accordingly, light is transmitted from the light source 20, through the fiber guide 22, and emitted out the distal end 14 of the endoscope 10 toward a targeted internal feature, such as tissue or an organ, of a body of a patient. As the light transmission pathway in such a configuration is relatively long, for example, the fiber guide 22 may be about 1.0 m to about 1.5 m in length, only about 15% (or less) of the light flux emitted from the light source 20 is outputted from the distal end 14 of the endoscope 10.

With reference to FIG. 2 and FIG. 3, the video system 30 is operatively connected to an image sensor 32 mounted to, or disposed within, the handle 18 of the endoscope 10 via a data cable 34. An objective lens 36 is disposed at the distal end 14 of the elongated body 12 of the endoscope 10 and a series of spaced-apart, relay lenses 38, such as rod lenses, are positioned along the length of the elongated body 12 between the objective lens 36 and the image sensor 32. Images captured by the objective lens 36 are forwarded through the elongated body 12 of the endoscope 10 via the relay lenses 38 to the image sensor 32, which are then communicated to the video system 30 for processing and output to the display device 40 via cable 39. The image sensor 32 is located within, or mounted to, the handle 18 of the endoscope 10, which can be up to about 30 cm away from the distal end 14 of the endoscope 10.

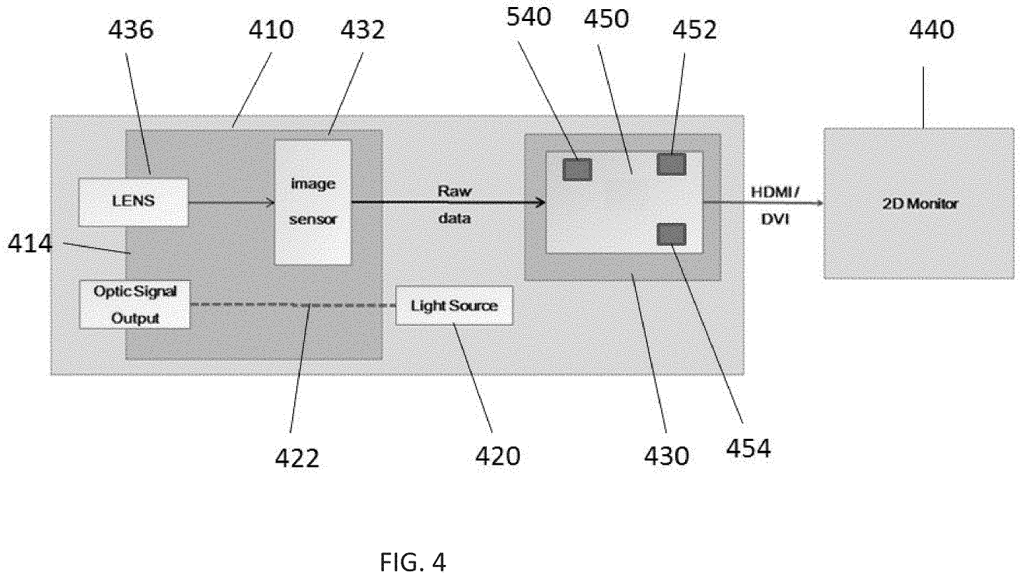

With reference to FIGS. 4-7, the flow diagrams include various blocks described in an ordered sequence. However, those skilled in the art will appreciate that one or more blocks of the flow diagram may be performed in a different order, repeated, and/or omitted without departing from the scope of the disclosure. The below description of the flow diagram refers to various actions or tasks performed by one or more video system 30, but those skilled in the art will appreciate that the video system 30 is exemplary. In various embodiments, the disclosed operations can be performed by another component, device, or system. In various embodiments, the video system 30 or other component/device performs the actions or tasks via one or more software applications executing on a processor. In various embodiments, at least some of the operations can be implemented by firmware, programmable logic devices, and/or hardware circuitry. Other implementations are contemplated to be within the scope of the disclosure.

Referring to FIG. 4, there is shown a schematic configuration of a system, which may be the endoscope system of FIG. 1 or may be a different type of system (e.g., visualization system, etc.). The system, in accordance with the disclosure, includes an imaging device 410, a light source 420, a video system 430, and a display device 440. The light source 420 is configured to provide light to a surgical site through the imaging device 410 via the fiber guide 422. The distal end 414 of the imaging device 410 includes an objective lens 436 for capturing the image at the surgical site. The objective lens 436 forwards the image to the image sensor 432. The image is then communicated to the video system 430 for processing. The video system 430 includes an imaging device controller 450 for controlling the endoscope and processing the images. The imaging device controller 450 includes processor 452 connected to a computer-readable storage medium or a memory 454 which may be a volatile type memory, such as RAM, or a non-volatile type memory, such as flash media, disk media, or other types of memory. In various embodiments, the processor 452 may be another type of processor such as, without limitation, a digital signal processor, a microprocessor, an ASIC, a graphics processing unit (GPU), field-programmable gate array (FPGA), or a central processing unit (CPU).

In various embodiments, the memory 454 can be random access memory, read-only memory, magnetic disk memory, solid state memory, optical disc memory, and/or another type of memory. In various embodiments, the memory 454 can be separate from the imaging device controller 450 and can communicate with the processor 452 through communication buses of a circuit board and/or through communication cables such as serial ATA cables or other types of cables. The memory 454 includes computer-readable instructions that are executable by the processor 452 to operate the imaging device controller 450. In various embodiments, the imaging device controller 450 may include a network interface 540 to communicate with other computers or a server.

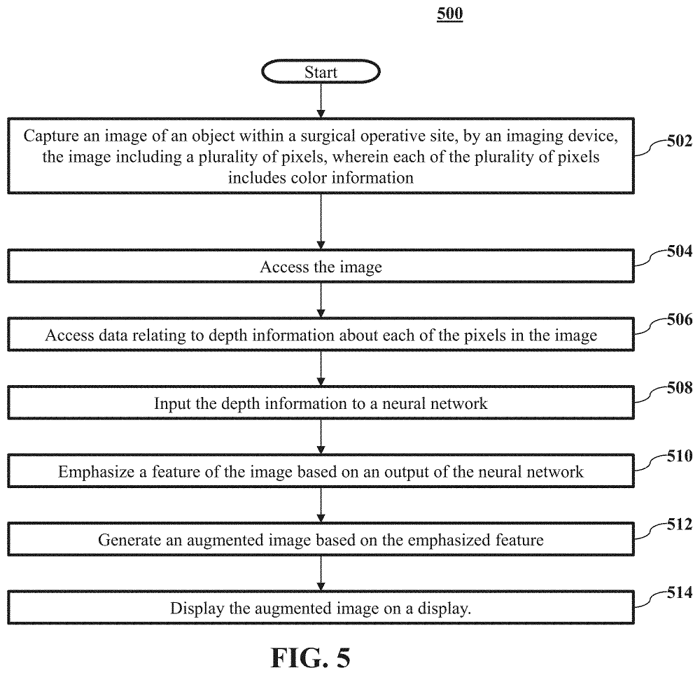

Referring now to FIG. 5, there is shown an operation for object enhancement in endoscopy images. In various embodiments, the operation of FIG. 5 can be performed by an endoscope system 1 described above herein. In various embodiments, the operation of FIG. 5 can be performed by another type of system and/or during another type of procedure. The following description will refer to an endoscope system, but it will be understood that such description is exemplary and does not limit the scope and applicability of the disclosure to other systems and procedures.

Initially, at step 502, an image of a surgical site is captured via the objective lens 36 and forwarded to the image sensor 32 of endoscope system 1. The term “image” as used herein may include still images or moving images (for example, video). The image includes a plurality of pixels, wherein each of the plurality of pixels includes color information. In various embodiments, the captured image is communicated to the video system 30 for processing. For example, during an endoscopic procedure, a surgeon may cut tissue with an electrosurgical instrument. When the image is captured, it may include objects such as the tissue and the instrument. For example, the image may contain several frames of a surgical site. At step 504, the video system 30 accesses the image for further processing.

At step 506, the video system 30 accesses data relating to depth information about each of the pixels in the image. For example, the system may access depth data relating to the pixels of an object in the image, such as an organ or a surgical instrument. In various embodiments, the image includes a stereographic image. In various embodiments, the stereographic image includes a left image and a right image. In various embodiments, the video system 30 may calculate depth information based on determining a horizontal disparity mismatch between the left image and the right image. In various embodiments, the depth information may include pixel depth. In various embodiments, the video system 30 may calculate depth information based on structured light projection.

At step 508, the video system 30 inputs the depth information to a neural network. In various embodiments, the neural network includes a convolutional neural network (CNN). CNNs are often thought of as operating on images, but they can just as well be configured to handle additional data inputs. The C in CNN stands for convolutional which is about applying matrix processing operations to localized portions of an image, and the results of those operations (which can involve dozens of different parallel and serial calculations) are sets of many features that are used to train neural networks. In various embodiments, additional information may be included in the operations that generate these features. In various embodiments, providing unique information that yields features that give the neural networks information that can be used to ultimately provide an aggregate way to differentiate between different data input to them. In various embodiments, the neural network may include a feed forward neural network, a radial bias neural network, a multilayer perceptron, a recurrent neural network, and/or a modular neural network.

In various embodiments, the depth information now associated with the pixels can be input to the image processing path to feed the neural network. At this point, the neural networks may start with various mathematical operations extracting and/or emphasizing 3D features. It is contemplated that the extraction of depth does not need to be real-time for training the neural networks. In various embodiments, a second source of enhancement of the images input to neural networks is to amplify the change in color of the pixels over time. This is a technique which can make subtle color changes or be magnified, for example, being able to discern one's pulse from the change in the color of a person's face as a function of cyclic cardiac output. In various embodiments, the change in tissue color as a result of various types of tool-tissue interactions such as grasping, cutting, and joining may be amplified. It is a function of the change in blood circulation, which would be cyclical as well as a result of tool effects on tissue. These enhanced time series videos can replace normal videos in the training and intraoperative monitoring process. It is contemplated that color change enhancement does not need to be real-time to train the networks.

In various embodiments, the neural network is trained based on tagging objects in training images, and wherein the training further includes augmenting the training images to include adding noise, changing colors, hiding portions of the training images, scaling of the training images, rotating the training images, and/or stretching the training images. In various embodiments, the training includes supervised, unsupervised, and/or reinforcement learning. It is contemplated that training images may be generated via other means that do not involve modifying existing images.

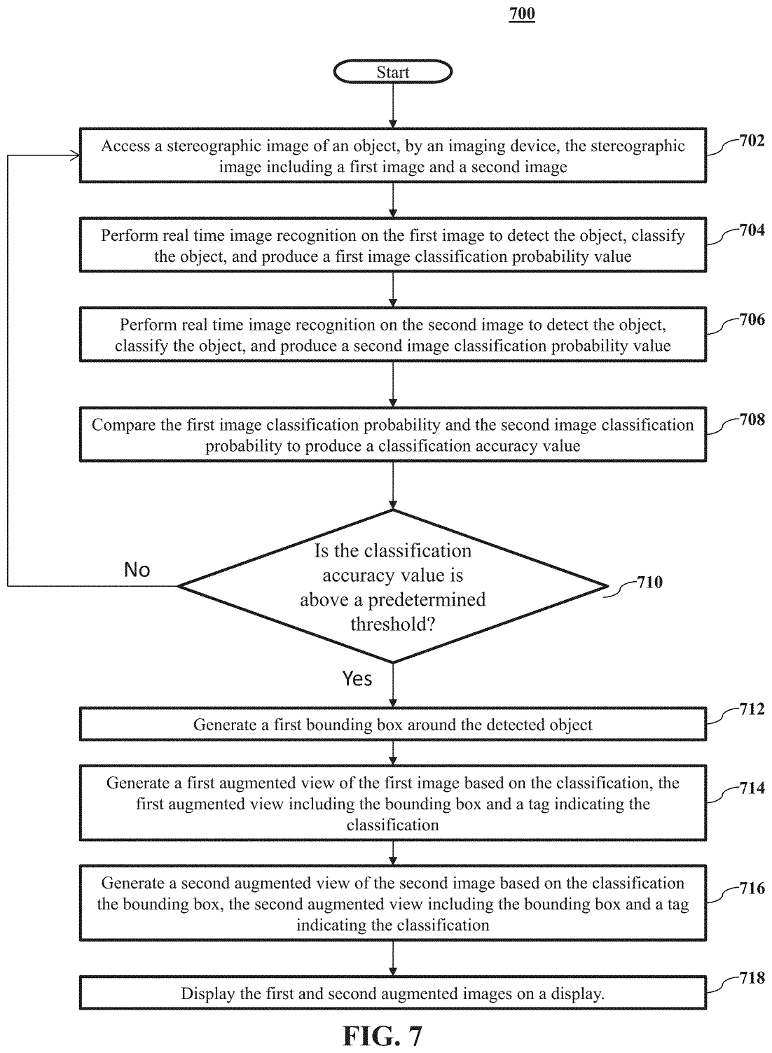

At step 510, the video system 30 emphasizes a feature of the image based on an output of the neural network. In various embodiments, emphasizing the feature includes augmenting a 3D aspect of the image, emphasizing a boundary of the object, changing the color information of the plurality of pixels of the object, and/or extracting 3D features of the object. In various embodiments, the video system 30 performs real-time image recognition on the augmented image to detect an object and classify the object. In various embodiments, the video system 30 processes a time series of the augmented image based on a learned video magnification, phase-based video magnification, and/or Eulerian video magnification. For example, the video system 30 may change the color of a surgical instrument to emphasize the boundary of the surgical instrument. In various embodiments, the enhanced image may be fed as an input into the neural network of FIG. 7 for additional object detection.

At step 512, the video system 30 generates an augmented image based on the emphasized feature. For example, the video system may generate an augmented image

At step 514, the video system 30 displays the augmented image on a display device 40. In various embodiments, the video system 30 performs tracking of the object based on an output of the neural network.

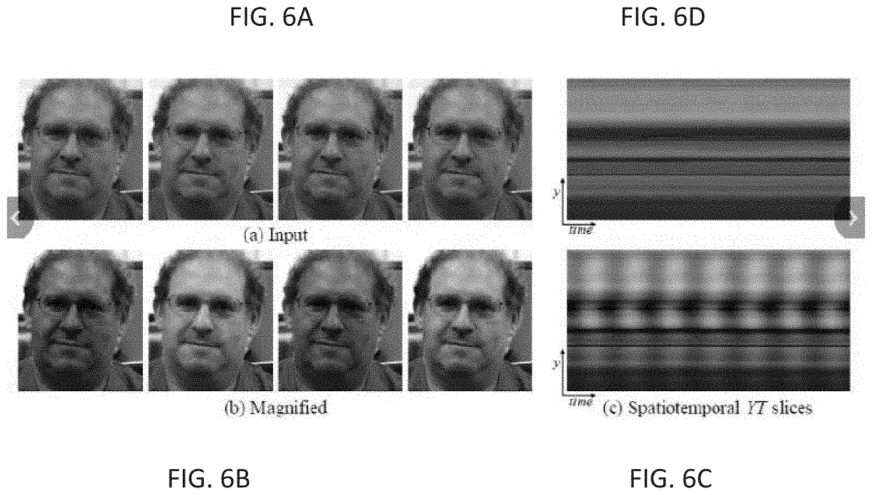

With reference to FIGS. 6A-6D, an exemplary image in accordance with the disclosure is shown. FIG. 6A shows four frames of an exemplary input image in accordance with the disclosure. FIG. 6B shows the four frames of the output image with the subject's pulse signal amplified in accordance with the disclosure. FIGS. 6C and 6D show an exemplary vertical scan line from the input image of FIG. 6B and output image FIG. 6A, respectively. The vertical scan line from the input and output images are plotted over time show how the method amplifies the periodic color variation. In FIG. 6D, the signal is nearly imperceptible. However, in FIG. 6C the color variation is readily apparent.

Referring now to FIG. 7, there is shown an operation for object detection in endoscopy images. In various embodiments, the operation of FIG. 7 can be performed by an endoscope system 1 described above herein. In various embodiments, the operation of FIG. 7 can be performed by another type of system and/or during another type of procedure. The following description will refer to an endoscope system, but it will be understood that such description is exemplary and does not limit the scope and applicability of the disclosure to other systems and procedures.

Initially, at step 702, a stereographic image of a surgical site is captured via the objective lens 36 and forwarded to the image sensor 32 of endoscope system 1. The term “image” as used herein may include still images or moving images (for example, video). The stereographic image including a first image and a second image (e.g., a left and a right image). The stereographic image includes a plurality of pixels, wherein each of the plurality of pixels includes color information. In various embodiments, the captured stereographic image is communicated to the video system 30 for processing. For example, during an endoscopic procedure a surgeon may cut tissue with an electrosurgical instrument. When the image is captured, it may include objects such as the tissue and the instrument.

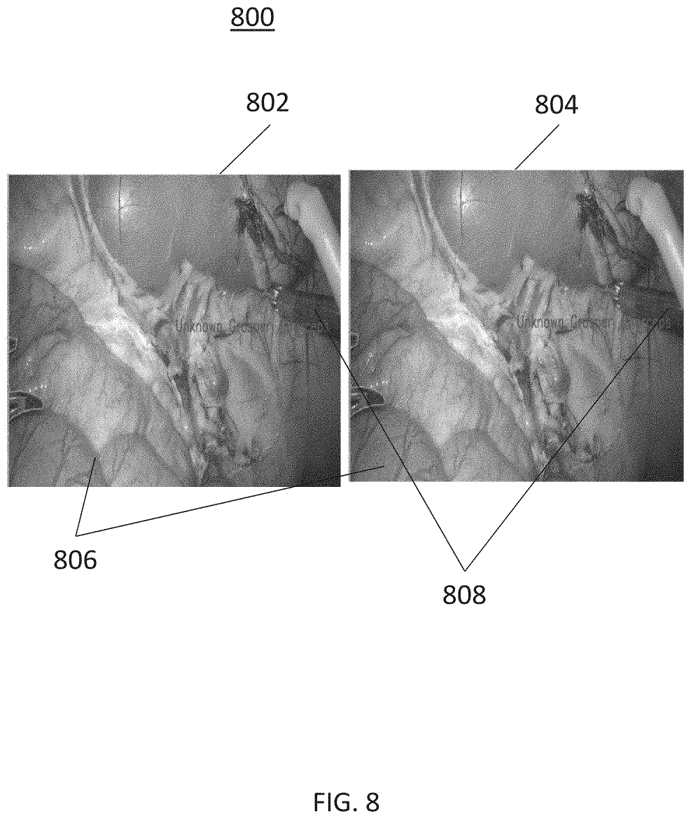

With reference to FIG. 8, a stereographic input image 800 of a surgical site is shown. The stereographic input image 800 includes a first image 802 (e.g., left image) and a second image 804 (e.g., right image). The first image 802 includes tissue 806 and an object 808. The second image 804 includes tissue 806 and an object 808. The object may include a surgical instrument, for example.

With continued reference to FIG. 7, at step 704, the video system 30 performs real-time image recognition on the first image to detect the object, classify the object and produce a first image classification probability value. For example, the video system 30 may detect a surgical instrument such as a stapler in the first image. For example, the detected object may include, but is not limited to, tissue, forceps, regular grasper, bipolar grasper, monopolar shear, suction, needle driver, and stapler. In various embodiments, to perform the real time image recognition the video system 30 may detect the object in the first image and detect the object in the second image. Next the video system 30 may generate a first silhouette of the object in the first image and generate a second silhouette of the object in the second image. Next, the video system 30 may compare the first silhouette to the second silhouette, and detect inconsistencies between the first silhouette and the second silhouette based on comparing the first silhouette and the second silhouette.

In various embodiments, to perform the real-time image recognition the video system 30 may detect the object based on a convolutional neural network. A convolutional neural network typically includes convolution layers, activation function layers, pooling (typically max-pooling) layers to reduce dimensionality without losing a lot of features. The detection may include initially generating a segmentation mask for the object, detecting the object and then classifying the object based on the detection.

In various embodiments, to perform the real-time image recognition, the video system 30 may detect the object based on a region based neural network. The video system 30 may detect the object by initially dividing the first image and second image into regions. Next, the video system 30 may predict bounding boxes for each region based on a feature of the object. Next, the video system 30 may predict an object detection probability for each region and weight the bounding boxes based on the predicted object detection probability. Next, the video system 30 may detect the object based on the bounding boxes and the weights and classify the object based on the detecting. In various embodiments, the region based or convolutional neural network may be trained based on tagging objects in training images. In various embodiments, the training may further include augmenting the training images to include adding noise, changing colors, hiding portions of the training images, scaling of the training images, rotating the training images, and/or stretching the training images.

Next, at step 706, the video system 30 performs real-time image recognition on the second image to detect the object, classify the object, and produce a second image classification probability value. For example, the video system 30 may detect a surgical instrument such as a stapler in the second image.

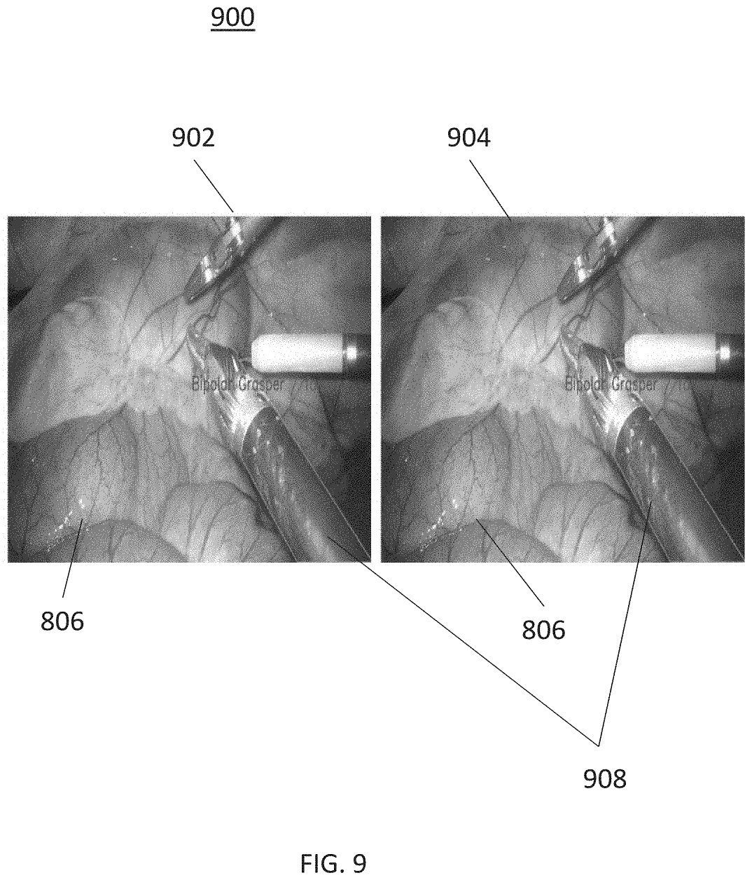

With reference to FIG. 9, a stereographic output image 900 of a surgical site is shown. The stereographic output image 900 includes a first image 902 (e.g., left image) and a second image 904 (e.g., right image). The first image includes tissue 806 and a detected object 908. The second image 904 includes tissue 806 and a detected object 908. For example, the video system 30 may classify the object 908 in the first image 902 as a bipolar grasper. For example, the video system 30 may classify the object 908 in the second image 904 as a bipolar grasper.

With continued reference to FIG. 7, at step 708, the video system 30 compare the first image classification probability value and the second image classification probability value to produce a classification accuracy value. For example, the first image classification probability value may be about 90% and the second image classification value may be around 87%, then for example, the video system 30 would produce a classification accuracy value of about 88.5%.

Next at step 710, the video system 30 determines whether the classification accuracy value is above a predetermined threshold. For example, the threshold may be about 80%. If the classification accuracy value is about 90%, then it would be above the predetermined threshold of 80%. If the video system 30 at step 710 determines whether the classification accuracy value is above a predetermined threshold, then at step 712, the video system 30 generates a first bounding box around the detected object.

Next at step 714, the video system 30 generates a first augmented view of the first image based on the classification. The first augmented view includes the bounding box and a tag indicating the classification. For example, the tag may be “stapler.”

Next at step 716, the video system 30 generates a second augmented view of the second image based on the classification of the bounding box. The augmented view including the bounding box and a tag indicating the classification. In various embodiments, the first and second augmented views each include an indication of the classification probability value.

Next at step 718, the video system 30 displays the first and second augmented images on a display device 40. In various embodiments, the video system 30 performs tracking of the object based on an output of the region based neural network.

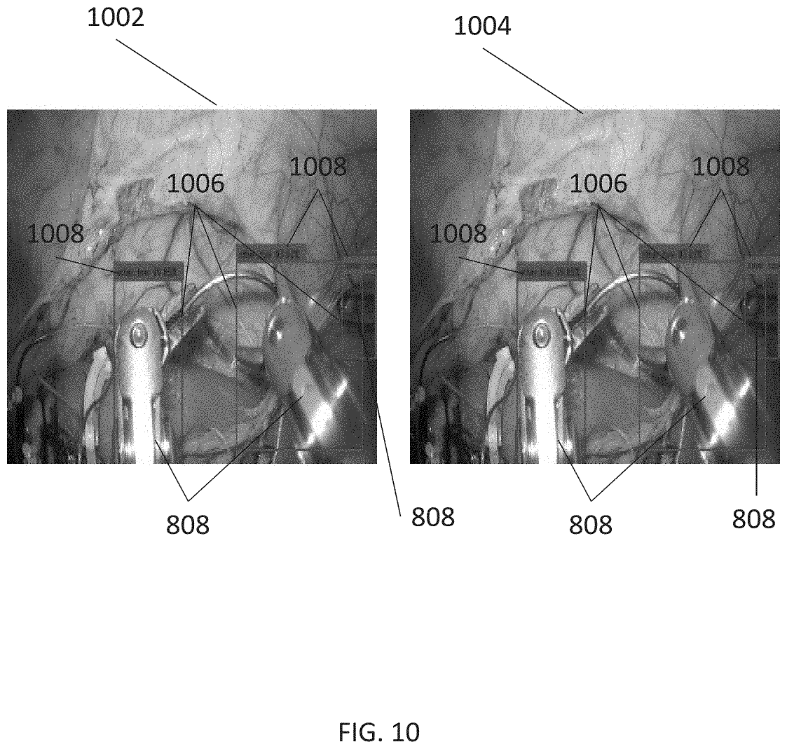

With reference to FIG. 10, the first augmented image 1002 and second augmented image 1004 are shown. The first augmented image 1002 includes a bounding box 1006 and a tag 1008. The tag 1008 may include the classification of the object and the classification probability value. For example, the classification of the object may be “other tool” and the classification probability value may be about 93%. It is contemplated that multiple objects may be detected and classified.

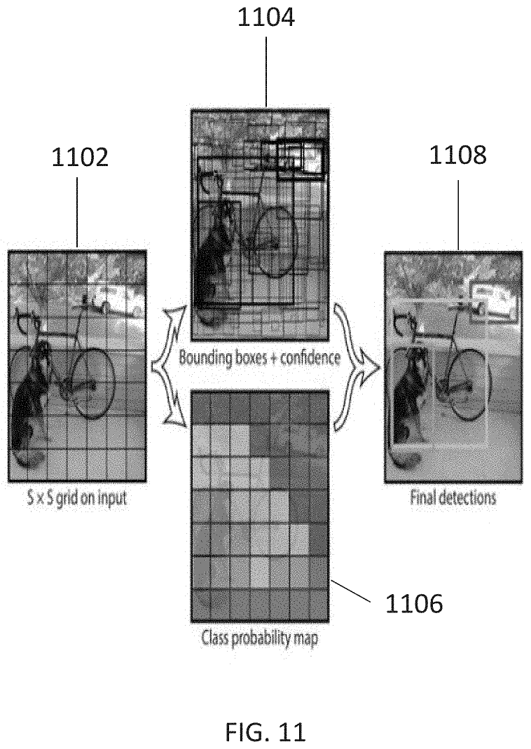

With reference to FIG. 11, an exemplary process for real-time image detection is shown. Initially, a neural network is applied to the full image. In various embodiments, the neural network then divides the image up into regions 1102 (e.g., an S×S grid). Next, the neural network predicts bounding boxes 1104 and probabilities 1106 for each of these regions. Then the bounding boxes 1104 are weighted by the predicted probabilities 1106 to output final detections 1108.

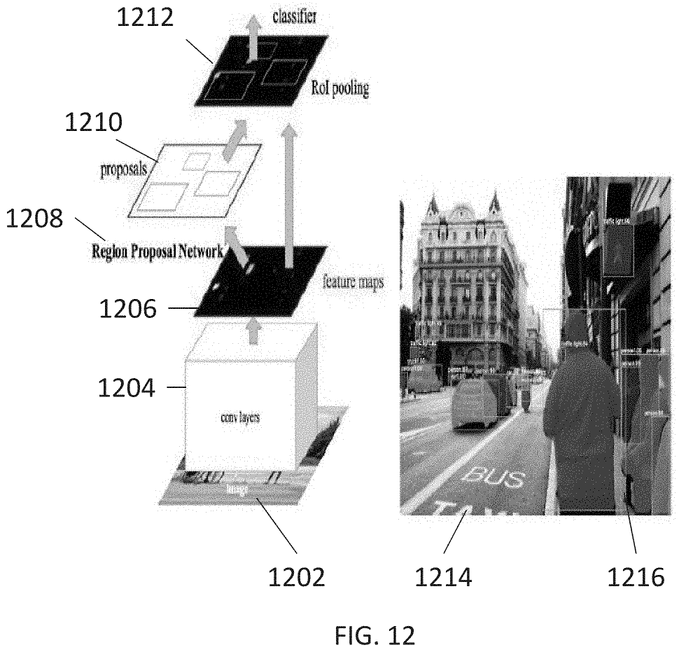

With reference to FIG. 12, a region proposal network for real-time image detection is shown. Initially, an image 1202 is input into a neural network 1204. In various embodiments, a convolutional feature map 1206 is generated by the last convolutional layer of the neural network 1204. In various embodiments, a region proposal network 1208 is slid over the convolutional feature map 1206 and generates proposals 1212 for the region of interest where the object lies. Generally, a region proposal network 1208 has a classifier and a regressor. A classifier determines the probability of a proposal having the target object. Regression regresses the coordinates of the proposals. Finally, the augmented image 1214 is output with bounding boxes 1216 and probabilities.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the disclosure in virtually any appropriately detailed structure. Like reference numerals may refer to similar or identical elements throughout the description of the figures.

The terms “artificial intelligence,” “data models,” or “machine learning” may include, but are not limited to, neural networks, convolutional neural networks (CNN), recurrent neural networks (RNN), generative adversarial networks (GAN), Bayesian Regression, Naive Bayes, nearest neighbors, least squares, means, and support vector regression, among other data science and artificial science techniques.

The phrases “in an embodiment,” “in embodiments,” “in some embodiments,” or “in other embodiments” may each refer to one or more of the same or different embodiments in accordance with the disclosure. A phrase in the form “A or B” means “(A), (B), or (A and B).” A phrase in the form “at least one of A, B, or C” means “(A); (B); (C); (A and B); (A and C); (B and C); or (A, B, and C).” The term “clinician” may refer to a clinician or any medical professional, such as a doctor, physician assistant, nurse, technician, medical assistant, or the like, performing a medical procedure.

The systems described herein may also utilize one or more controllers to receive various information and transform the received information to generate an output. The controller may include any type of computing device, computational circuit, or any type of processor or processing circuit capable of executing a series of instructions that are stored in a memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, causes the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Any of the herein described methods, programs, algorithms, or codes may be contained on one or more machine-readable media or memory. The term “memory” may include a mechanism that provides (for example, stores and/or transmits) information in a form readable by a machine such a processor, computer, or a digital processing device. For example, a memory may include a read only memory (ROM), random access memory (RAM), magnetic disk storage media, optical storage media, flash memory devices, or any other volatile or non-volatile memory storage device. Code or instructions contained thereon can be represented by carrier wave signals, infrared signals, digital signals, and by other like signals.

It should be understood that the foregoing description is only illustrative of the disclosure. Various alternatives and modifications can be devised by those skilled in the art without departing from the disclosure. Accordingly, the disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

1. A system for object enhancement in endoscopy images, comprising:
a light source configured to provide light within a surgical operative site;
an imaging device configured to acquire images;
an imaging device control unit configured to control the imaging device, the imaging device control unit including:
a processor; and
a memory storing instructions which, when executed by the processor, cause the system to:
capture an image of an object within the surgical operative site, by the imaging device, the image including a plurality of pixels, wherein each of the plurality of pixels includes color information;
access the image;
access data relating to depth information about each of the pixels in the image;
input the depth information to a machine learning algorithm;
emphasize a feature of the image based on an output of the machine learning algorithm;
generate an augmented image based on the emphasized feature; and
display the augmented image on a display.

2. The system of claim 1, wherein emphasizing the feature includes at least one of: augmenting a 3D aspect of the image, emphasizing a boundary of the object, changing the color information of the plurality of pixels of the object, or extracting 3D features of the object.

3. The system of claim 1, wherein the instructions, when executed, further cause the system to perform real-time image recognition on the augmented image to detect an object and classify the object.

4. The system of claim 1, wherein the image includes a stereographic image, and wherein the stereographic image includes a left image and a right image,
wherein the instructions, when executed, further cause the system to calculate depth information based on determining a horizontal disparity mismatch between the left image and the right image, and
wherein the depth information includes pixel depth.

5. The system of claim 1, wherein the instructions, when executed, further cause the system to calculate depth information based on structured light projection,
wherein the depth information includes pixel depth.

6. The system of claim 1, wherein the machine learning algorithm includes at least one of a convolutional neural network, a feed forward neural network, a radial bias neural network, a multilayer perceptron, a recurrent neural network, or a modular neural network.

7. The system of claim 1, wherein the machine learning algorithm is trained based on tagging objects in training images, and wherein the training further includes augmenting the training images to include at least one of adding noise, changing colors, hiding portions of the training images, scaling of the training images, rotating the training images, or stretching the training images.

8. The system of claim 7, wherein the training includes at least one of supervised, unsupervised, or reinforcement learning.

9. The system of claim 1, wherein the instructions, when executed, further cause the system to:
process a time series of the augmented image based on at least one of a learned video magnification, phase-based video magnification, or Eulerian video magnification.

10. The system of claim 9, wherein the instructions, when executed, further cause the system to:
perform tracking of the object based on an output of the machine learning algorithm.

11. A computer-implemented method of object enhancement in endoscopy images, comprising:
capturing an image of an object within a surgical operative site, by an imaging device, the image including a plurality of pixels, wherein each of the plurality of pixels includes color information;
accessing the image;
accessing data relating to depth information about each of the pixels in the image;
inputting the depth information to a machine learning algorithm;
emphasizing a feature of the image based on an output of the machine learning algorithm;
generating an augmented image based on the emphasized feature; and
displaying the augmented image on a display.

12. The computer-implemented method of claim 11, wherein emphasizing the feature includes at least one of: augmenting a 3D aspect of the image, emphasizing a boundary of the object, changing the color information of the plurality of pixels of the object, or extracting 3D features of the object.

13. The computer-implemented method of claim 11, wherein the computer-implemented method further comprises performing real-time image recognition on the augmented image to detect an object and classify the object.

14. The computer-implemented method of claim 11, wherein the image includes a stereographic image, and wherein the stereographic image includes a left image and a right image,
wherein the computer-implemented method further comprises calculating depth information based on determining a horizontal disparity mismatch between the left image and the right image, and
wherein the depth information includes pixel depth.

15. The computer-implemented method of claim 11, wherein the computer-implemented method further comprises calculating depth information based on structured light projection,
wherein the depth information includes pixel depth.

16. The computer-implemented method of claim 11, wherein the machine learning algorithm includes at least one of a convolutional neural network, a feed forward neural network, a radial bias neural network, a multilayer perceptron, a recurrent neural network, or a modular neural network.

17. The computer-implemented method of claim 11, wherein the machine learning algorithm is trained based on tagging objects in training images, and wherein the training further includes augmenting the training images to include at least one of adding noise, changing colors, hiding portions of the training images, scaling of the training images, rotating the training images, or stretching the training images.

18. The computer-implemented method of claim 11, wherein the computer-implemented method further comprises processing a time series of the augmented image based on at least one of a learned video magnification, phase-based video magnification, or Eulerian video magnification.

19. The computer-implemented method of claim 18, wherein the computer-implemented method further comprises performing tracking of the object based on an output of the machine learning algorithm.

20. A non-transitory storage medium that stores a program causing a computer to execute a computer-implemented method of object enhancement in endoscopy images, the computer-implemented method comprising:
capturing an image of an object within a surgical operative site, by an imaging device, the image including a plurality of pixels, wherein each of the plurality of pixels includes color information;
accessing the image;
accessing data relating to depth information about each of the pixels in the image;
inputting the depth information to a machine learning algorithm;
emphasizing a feature of the image based on an output of the machine learning algorithm;
generating an augmented image based on the emphasized feature; and
displaying the augmented image on a display.

21-39. (canceled)
